(19) United States
(12) Patent Application Publication
(10) Pub. No.: US 2012/0058205 A1
(43) Pub. Date: Mar. 8, 2012

(54) COMPOSITIONS AND TREATMENT REGIMES FOR REDUCING INFLAMMATION, END-ORGAN INJURY, AND/OR SYSTEMIC ENDOTOXEMIA

(76) Inventor: Walter Howard Peschel, Missoula, MT (US)

(21) Appl. No.: 13/255,089

(22) PCT Filed: Mar. 4, 2010
(86) PCT No.: PCT/US10/26283

§ 371 Date: Oct. 25, 2011

Related U.S. Application Data
(60) Provisional application No. 61/157,370, filed on Mar. 4, 2009.

(51) Int. Cl. A61K 31/585; A61P 3/06; A61P 13/12; A61P 37/00; A61P 27/02; A61K 33/06; A61P 29/00

(52) U.S. Cl. 424/682; 514/171

(57) ABSTRACT

The invention relates to pharmaceutical compositions and treatment regimes useful in treating one or more of the following conditions: inflammation, kidney disease, eye disease, end-organ injury, systemic endotoxemia, and/or vitamin D resistance. The compositions and treatment regimes are also useful in reducing elevated CRP levels and/or elevated pro-inflammatory cytokines.

### CROSS-REFERENCE TO RELATED APPLICATIONS

This application claims the benefit of U.S. Provisional Application No. 61/157,370, filed Mar. 4, 2009, the content of which is expressly incorporated herein in its entirety by reference thereto.

### TECHNICAL FIELD

The invention relates to pharmaceutical compositions and treatment regimes useful in treating one or more of the following conditions in a subject: inflammation, kidney disease, eye disease, end-organ injury after surgery, systemic endotoxemia, and/or vitamin D resistance.

### BACKGROUND OF THE INVENTION

I. Negative Physiological Effects Following Coronary Artery Bypass Surgery or other Ischemic Related Events:

Coronary artery bypass surgery is associated with almost immediate systemic endotoxemia and release of pro-inflammatory cytokines. The trigger for these events is multi-factorial, and the resulting systemic inflammatory response syndrome (SIRS) results in dysfunction and microcirculation, decreased perfusion of organs, and subsequent multiple organ dysfunction (MOD).

The dysfunction is the result of dysregulated immune processes. The pathology spares no tissue but predominates in the CNS, heart, lung, renal, and GI systems.

At the center of the micro-circulatory dysfunction is a dysregulation of free radicals. Free radicals are atoms that contain one or more unpaired electrons in the outer shell. They are short-live and unstable, being highly reactive, trying to trade their unpaired electrons and hydrogen ions and other adjacent molecules, resulting in denaturing of proteins, peroxidation of lipids, and fracturing of strands of DNA. The principal free radicals are nitric oxide and members of the reactive oxygen species, superoxide and H2O2.

The beneficial effects of nitric oxide (NO and reactive oxygen species (ROS) occur transiently at low to moderate concentrations and mediate physiologic roles in defense against infection, cell-signaling pathways, and induction or prevention of cellular proliferation and apotosis. They are formed continuously in small amounts in normal cellular metabolism and are normally inactivated by endogenous scavenger mechanisms after they have functioned. Under conditions of ischemia and inflammation, free radicals can be overproduced or under-inactivated resulting in their accumulation, becoming mechanisms of disease.

A need exist to reduce or eliminate the negative systemic events that occur after coronary artery bypass surgery, or other ischemic related events. The present invention provides direction in meeting this need.

### II. The Renin-Angiotensin System (RAS)

A. The Circulatory RAS

The renin-angiotensin system (RAS) or the renin-angiotensin-aldosterone system (RAAS) is a hormone system that regulates water (fluid) balance and blood pressure.

When blood volume decreases, the kidneys secrete the enzyme renin. Angiotensinogen, an inactive molecule secreted by the liver, binds to renin and is converted to the inactive molecule Angiotensin I. Angiotensin-converting enzyme (ACE) converts Angiotensin I into Angiotensin II. Angiotensin II is an active hormone that can bind to two different receptors (AT₁ and/or AT₂) and exert biological effects in various tissues. Angiotensin causes blood vessels to constrict, resulting in increased blood pressure.

When Angiotensin II binds to an AT₂ receptor, it can stimulate the production of nitric oxide (NO), which dilates the arteries and improves microcirculation, which in turn allows blood to flow more freely and prevents clotting in the vessels.

Angiotensin II can also bind to an AT₁ receptor. These receptors mediate several physiological responses which are critical to cardiovascular and renal function. Binding stimulates vasoconstriction, raising blood pressure. Angiotensin II also stimulates aldosterone synthesis by the adrenal gland. This results in sodium and water reabsorption which in turn increases blood volume, and ultimately, blood pressure.

If the circulatory renin-angiotensin-aldosterone system is overactive, blood pressure will be too high. There are many drugs that interrupt different steps in this system to lower blood pressure. These drugs are often primary means to control high blood pressure (hypertension), heart failure, kidney failure, and harmful effects of diabetes.

B. Tissue RAS

Subsequent studies on people thought to have an overactive circulatory RAS, however, revealed low renin and aldosterone levels. Further findings revealed a tissue based RAS, which has revolutionized research in this area. Overactive tissue RAS has increasingly been linked to harmful inflammation.

Early research confirmed that renin receptors line tissue membranes throughout the body. Moreover, the renin receptors predominated in the tissues most susceptible to inflammation, such as the kidneys, eyes, and heart. Prorenin, previously assumed to be an inactive molecule, (e.g. because it cannot convert angiotensinogen to angiotensin I in vitro), became the subject of renewed interest. Researchers correlated elevated prorenin levels in diabetics with increased progression of the disease. Similarly, elevated prorenin levels preceded microvascular disease of the eyes and kidneys and harmful inflammation.

Although both renin and prorenin can bind to the renin receptor, prorenin does so at a much higher rate. This is because, as compared to renin, prorenin as it has a much higher affinity for the receptor and is present in the tissues at much greater levels.

### SUMMARY OF THE INVENTION

The present invention is directed to a pharmaceutical composition comprising an effective amount of a fibrate anti-hyperlipidemic agent, a MG-CoA reductase inhibitor, an aldosterone antagonist, and optionally a pharmaceutically acceptable carrier. Advantageously, one or more other pharmacologically active ingredients may be included. For example, in certain preferred forms, the composition further comprises a thiazolidinedione (TZD), a Angiotensin II Receptor Antagonists (AIIRA), Vitamin D and/or calcium. Preferably the fibrate anti-hyperlipidemic agent is gemfibrozil, the MG-CoA reductase inhibitor is a statin, and/or the aldosterone antagonist is a spironolactone or eplerenone. Vitamin D can be in any form, however, Vitamin D₂ and Vitamin D₃ are preferred forms, and calcium is preferably in a bioactive form.

The composition of invention can be used as a medicament for therapy, for example, to treat inflammation, kidney disease, eye disease, elevated CRP levels, end-organ injury after surgery, systemic endotoxemia, and/or elevated pro-inflammatory cytokines.

Furthermore, in certain embodiments, the pharmaceutical composition is preferably formulated for oral administration, example, in a single daily oral dose, twice daily oral dose, or thrice daily oral dose.

In a particular embodiment, the present invention is a pharmaceutical treatment regime for reducing inflammation in a subject. The regime typically comprises the steps of administering to a subject in need thereof at least the following pharmacologically active ingredients within a 24 hour period: a fibrate anti-hyperlipidemic agent, a MG-CoA reductase inhibitor, an aldosterone antagonist, and a pharmaceutically acceptable carrier, wherein the pharmacologically active ingredients are in an amount sufficient to reduce inflammation, more preferably in an amount sufficient to reduce the level of at least one pro-inflammatory marker in the subject, e.g., prorenin, renin, renin mRNA, aldosterone, and TGF-β.

Surprisingly, the pharmaceutical treatment regime of the invention disclosed herein, can be used to reduce systemic endotoxemia and/or pro-inflammatory cytokines; reduce severity of eye disease; reduce severity of kidney disease; and/or reduce end-organ injury after surgery; e.g., a cardiopulmonary bypass surgery.

The present invention is further directed to a regime to lower the C-reactive protein (CRP) level in a patient's blood.

### DETAILED DESCRIPTION OF THE PREFERRED EMBODIMENTS

### I. The Composition

The present invention is directed to pharmaceutical compositions useful in preventing and treating inflammation and certain diseases. The compositions of the invention are useful in treating vitamin D resistance, kidney disease, eye disease and systemic endotoxemia, for example. The compositions have also been found to be useful in reducing elevated CRP levels and/or elevated pro-inflammatory cytokines.

In a preferred embodiment, the pharmaceutical composition comprises an effective amount of a fibrate anti-hyperlipidemic agent, a MG-CoA reductase inhibitor, an aldosterone antagonist, and optionally a pharmaceutically acceptable carrier. The fibrate anti-hyperlipidemic agent is typically between 100-1200 mg; the MG-CoA reductase inhibitor is typically between 5-80 mg; and the aldosterone antagonist is typically between 5-100 mg.

Preferably, the fibrate anti-hyperlipidemic agent is gemfibrozil, the MG-CoA reductase inhibitor is a statin, and the aldosterone antagonist is a spironolactone or eplerenone; and the gemfibrozil is between 400-800 mg; the statin is between 10-30 mg; the spironolcatone is between 10-75 mg or eplereonone 15-70 mg.

Advantageously, one or more additional pharmacologically active ingredients may be included, for example, a thiazolidinedione (TZD) or an Angiotensin II Receptor Antagonists (AIIRA). In a preferred embodiment, that the TZD is pioglitazone and the AIIRA is a telemisartan, for example, pioglitazone between 5-60 mg, more preferably between 35-55 mg.

In a specific non-limiting embodiment, the fibrate anti-hyperlipidemic agent is 5-(2,5-dimethylphenoxy)-2,2-dimethylpentanoic acid;, the MG-CoA reductase inhibitor is (3R,5R)-7-[2-(4-fluorophenyl)-3-phenyl-4-(phenylcarbamoyl)-5-(propan-2-yl)-1H-pyrrol-1-yl]-3,5-dihydroxyheptanoic acid; and the aldosterone antagonist is either 7α-Acetylthio-3-oxo-17α-pregn-4-ene-21,17-carbolactone or pregn-4-ene-7,21-dicarboxylic acid, 9,11-epoxy-17-hydroxy-3-oxo, γ-lactone, methyl ester (7α, 11α, 17α). The composition may also further include 5-(4-[2-(5-ethylpyridin-2-yl)ethoxy]benzyl)thiazolidine-2,4-dione.

Another preferred embodiment is directed to a kit, wherein the kit comprises an effective amount of the following pharmacologically active ingredients: a fibrate anti-hyperlipidemic agent, a MG-CoA reductase inhibitor, an aldosterone antagonist and optionally a pharmaceutically acceptable carrier. Preferably, the kit further comprises a thiazolidinedione (TZD) or a Angiotensin II Receptor Antagonists (AIIRA). More preferably, the kit further comprises Vitamin D and/or calcium. Most preferably, the kit further comprises instructions to administer the pharmacologically active ingredients to a subject within a 24 hour period.

A. Vitamin D and/or Calcium

Preferably, the composition further comprises Vitamin D and/or calcium. In a specific non-limiting embodiment, the Vitamin D is selected from the group consisting of Vitamin D₂ and Vitamin D₃. Preferably, the Vitamin D₃ is between 1,000 IU and 50,000 IU. More preferably, the amount of Vitamin D₃ is between 5,000 IU and 20,000 IU. Alternatively, the Vitamin D can be in the form Vitamin D₂, in an amount between 2,500 IU and 75,000 IU. Preferably, the Vitamin D₂ is between 25,000 IU and 42,500 IU. In a preferred embodiment, the calcium is between 500 mg and 4,000 mg. More preferably, the calcium is between 1,000 mg and 3,000 mg.

The compositions and/or components of the present invention can be used in a method of lowering the C-Reactive Protein (CRP) level in a patient's blood, preferably a human. The method comprises administering to the patient the pharmaceutical compositions described above, or the components of the composition separately during a 24 hour period. The amount to be administered must be sufficient to lower the CRP level in the patient's blood.

### II. The Pharmaceutical Treatment Regime

The present invention is also directed to pharmaceutical treatment regimes useful in treating one or more of the following conditions: inflammation, kidney disease, eye disease, end-organ injury after surgery, systemic endotoxemia, and/or vitamin D resistance. The compositions are also useful in reducing elevated CRP levels and/or elevated pro-inflammatory cytokines.

A preferred treatment regime comprises administering to a subject in need thereof at least the following pharmacologically active ingredients within a 24 hour period: a fibrate anti-hyperlipidemic agent, a MG-CoA reductase inhibitor, an aldosterone antagonist, and optionally a pharmaceutically acceptable carrier. The ingredients may be administered in one, two or more daily dosages. Preferably the dosages are in an oral form, however, other dosage forms are well within the scope of the invention.

The fibrate anti-hyperlipidemic agent used is typically between 100-1200 mg; the MG-CoA reductase inhibitor is between 5-80 mg; and the aldosterone antagonist is between 5-100 mg. Preferably, the fibrate anti-hyperlipidemic agent used is gemfibrozil, the MG-CoA reductase inhibitor is a statin, and the aldosterone antagonist is a spironolactone or eplerenone; and the gemfibrozil is between 400-800 mg; the statin is between 10-30 mg; the spironolcatone is between 10-75 mg or eplereonone 15-70 mg.

Advantageously, one or more additional pharmacologically active ingredients may be included in the regime as well, for example, a thiazolidinedione (TZD) or an Angiotensin II Receptor Antagonists (AIIRA). In a preferred embodiment, the TZD is pioglitazone and the AIIRA is a telemisartan, for example, pioglitazone between 5-60 mg, more preferably between 35-55 mg.

In a specific non-limiting embodiment the fibrate anti-hyperlipidemic agent is 5-(2,5-dimethylphenoxy)-2,2-dimethylpentanoic acid; the MG-CoA reductase inhibitor is (3R,5R)-7-[2-(4-fluorophenyl)-3-phenyl-4-(phenylcarbamoyl)-5-(propan-2-yl)-1H-pyrrol-1-yl]-3,5-dihydroxyheptanoic acid; and the aldosterone antagonist is either 7α-Acetylthio-3-oxo-17α-pregn-4-ene-21,17-carbolactone or pregn-4-ene-7,21-dicarboxylic acid, 9,11-epoxy-17-hydroxy-3-oxo, γ-lactone, methyl ester (7α, 11α, 17α). The composition may also further include 5-(4-[2-(5-ethylpyridin-2-yl)ethoxy]benzyl)thiazolidine-2,4-dione.

Preferably, the regime further comprises administration of Vitamin D and/or calcium. In a specific non-limiting embodiment, the Vitamin D used in the regime is selected from the group consisting of: Vitamin D₂ and Vitamin D₃. Preferably, the Vitamin D₃ is between 1,000 IU and 30,000 IU. More preferably, the amount of Vitamin D₃ is between 5,000 IU and 20,000 IU. Alternatively, the Vitamin D can be in the form Vitamin D₂, in an amount between 2,500 IU and 75,000 IU. Preferably, the Vitamin D₂ is between 25,000 IU and 42,500 IU. In a preferred embodiment, the calcium is between 500 mg and 4,000 mg. More preferably, the calcium is between 1,000 mg and 3,000 mg.

Another preferred embodiment is directed to a kit, wherein the kit comprises an effective amount of the following pharmacologically active ingredients: a fibrate anti-hyperlipidemic agent, a MG-CoA reductase inhibitor, an aldosterone antagonist and optionally a pharmaceutically acceptable carrier, and wherein the kit further comprises instructions describing any of the pharmaceutical treatment regimes herein. Preferably, the kit further comprises a thiazolidinedione (TZD) or a Angiotensin II Receptor Antagonists (AIIRA). More preferably, the kit further comprises Vitamin D and/or calcium. Most preferably, the kit further comprises instructions to administer the pharmacologically active ingredients to a subject within a 24 hour period.

A. Treatment of Inflammation and Reduction of Proinflammatory Markers

In a preferred embodiment, the pharmaceutical treatment regime is used to treat a subject suffering from inflammation. In this embodiment, the active ingredients are administered in an amount sufficient to reduce inflammation in the subject.

Typically, the treatment regime reduces the levels of one or more proinflammatory markers in a subject, such as, proinflammatory markers prorenin, renin, renin and (pro)renin mRNA, aldosterone, and TGF-β, pro-inflammatory cytokines, or other pro-inflammatory.

In a preferred embodiment, the treatment regime reduces the level of one or more pro-inflammatory marker in a subject by at least 10%. Preferably, the treatment regime reduces the level of any pro-inflammatory marker in a subject by at least 25%, more preferably by at least 60%.

B. Lowering C-Reactive Protein (CRP) levels

In another embodiment, the treatment regime reduces the level of CRP in a patient's blood. Preferably, the treatment regime reduces the level of CRP in a patient's blood by at least 40%, more preferably by at least 50%, and most preferably by at least 75%.

C. Reducing End-Organ Injury after Surgery and Treating Systemic Endotoxemia

In yet another embodiment, the invention is directed to a treatment regime for reducing end-organ injury after surgery, e.g., a cardiopulmonary bypass surgery, treating systemic endotoxemia and/or reducing pro-inflammatory cytokines in a subject.

D. Reducing Eye Disease, Kidney Disease, and Reducing Vitamin D Resistance.

The treatment regime is also useful in reducing eye disease and/or kidney disease and/or Vitamin D resistance. Preferably, the reduction in Vitamin D resistance allows administration to a subject of the pharmaceutical composition without a corresponding increase in Vitamin D toxicity. More preferably, the absence of Vitamin D toxicity remains even when the composition comprises Vitamin D in amounts up to 50,000 IU (Vitamin D₃) or 125,000 IU (Vitamin D₂).

### III. Pharmaceutical Preparations and Administrations:

The pharmaceutical compositions of the invention can take a variety of forms adapted to the chosen route of administration as discussed above. Those skilled in the art will recognize various synthetic methodologies that may be employed to prepare non-toxic pharmaceutically acceptable compositions of the compounds described herein. Those skilled in the art will recognize a wide variety of non-toxic pharmaceutically acceptable solvents that may be used to prepare solvates of the compounds of the invention, such as water, ethanol, mineral oil, vegetable oil, and dimethylsulfoxide.

The compositions of the invention may be administered orally, topically (e.g., to prevent restenosis in sirolimus-coated stents), parenterally, by inhalation or spray or rectally in dosage unit formulations containing conventional non-toxic pharmaceutically acceptable carriers, adjuvants and vehicles. It is further understood that the best method of administration may be a combination of methods. Oral administration in the form of a pill, capsule, elixir, syrup, lozenge, troche, or the like is particularly preferred. The term parenteral as used herein includes subcutaneous injections, intradermal, intravascular (e.g., intravenous), intramuscular, spinal, intrathecal injection or like injection or infusion techniques.

The pharmaceutical compositions containing compounds of the invention are preferably in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropyl-methylcellulose, sodium alginate, polyvinylpyrrolidone, gum tragacanth and gum acacia; and dispersing or wetting agents, which may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water to provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth; naturally-occurring phosphatides, for example soy bean, lecithin, and esters or partial esters derived from fatty acids and hexitol; anhydrides, for example sorbitan monooleate; and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The composition of the invention may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

Alternatively, the compositions can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

In one embodiment, the compositions are administered in conjunction with an endovascular device. Preferably the device is a stent, stent graft, a graft, a graft connector, a guide wire, a catheter or a catheter pump, or a balloon catheter and the cardiovascular disease being treated is stenosis or restenosis.

For administration to non-human animals, the composition containing the therapeutic compound may be added to the animal's feed or drinking water. Also, it will be convenient to formulate animal feed and drinking water products so that the animal takes in an appropriate quantity of the compound in its diet. It will further be convenient to present the compound in a composition as a premix for addition to the feed or drinking water. The composition can also added as a food or drink supplement for humans.

Frequency of dosage may also vary depending on the compound used and the particular disease treated. However, for treatment of most disorders, a dosage regimen of 3 times daily or less is preferred. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

Preferred compounds of the invention will have desirable pharmacological properties that include, but are not limited to, oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Toxicity to cultured hepatocyctes may be used to predict compound toxicity. Penetration of the blood brain barrier of a compound in humans may be predicted from the brain levels of laboratory animals that receive the compound intravenously.

Serum protein binding may be predicted from albumin binding assays. Such assays are described in a review by Oravcova, et al. (Journal of Chromatography B (1996) volume 677, pages 1-27).

Compound half-life is inversely proportional to the frequency of dosage of a compound. In vitro half-lives of compounds may be predicted from assays of microsomal half-life as described by Kuhnz and Gieschen (Drug Metabolism and Disposition, (1998) volume 26, pages 1120-1127).

It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

The amount of the composition required for use in treatment will vary not only with the particular compound selected but also with the route of administration, the nature of the condition being treated and the age and condition of the patient and will ultimately be at the discretion of the attendant physician or clinician.

### Methods

The clinical methods for measuring and monitoring disease regression, such as: eye and kidney disease, reduction in inflammation, reduction in pro-inflammatory markers (including CRP), and reduction in end-organ injury, are well known to one skilled in the art. The following specific, non-limiting methods are exemplary in nature and do not preclude the use of other methods for monitoring disease regression and reduction in inflammation.

Measuring Renin Levels

Two primary assays have long predominated for measuring renin. The first relies on the enzymatic activity of renin. The predominant assay has long been the plasma renin activity (PRA) assay. It is performed by incubating plasma in the presence of inhibitors of Angiotensin I-degrading enzymes. The presence of Angiotensin I is an index of renin activity. Its production depends not only on the amount of renin, but also on the angiotensinogen concentration in plasma. This concentration is generally 1000 to 1500 nM, (e.g. close to the Michaelis constant (Km)). Consequently, care must be taken not to dilute the sample.

Exogenous substrate should be added in saturating quantities to make the assay independent of angiotensinogen concentration (in other words, to measure plasma renin concentration (PRC) rather than PRA). Because human angiotensinogen is not readily available, plasma from nephrectomized sheep may be used instead. Under saturating conditions, Angiotensin I production is directly proportional to the concentration of renin. In general, PRC correlates well with PRA. However, there are some exceptions in pregnant women and women on contraceptive pills.

The second type of renin assay is a direct immunoassay. At least three assays are widely used: an immunoradiometric assay (IRMA) by Cis Bio (See J. Clin Invest. 1984; 74: 723-735), and 2 assays by Nichols Diagnostics (See Clin Chem. 2004; 50: 2111-2116), an IRMA and a chemoluminometric assay that runs on an automated platform. All 3 assays use an immobilized capture antibody that binds both renin and prorenin. The second developing antibody is specific for renin and is labeled by either radioactive iodine for the IRMA or acridinium for the chemoluminometric assay.

The results of direct immunoassays of renin are identical to those of the enzymatic renin concentration assays (with added angiotensinogen) provided they have been calibrated with the same standard. The WHO has kept a reference preparation since 1974, consisting of a partially purified kidney renin that is defined by its enzymatic activity and therefore expressed in units per liter. Correlation between PRA and renin immunoassays is usually accurate, and for clinical purposes both assays may be used.

All renin assays may overestimate renin because of the presence of cryoactivated prorenin. Samples should therefore never be left on ice for prolonged periods of time. The Nichols IRMA suffered from overestimation of renin through comeasurement of prorenin, but this was solved by shorter incubation at higher temperature.

Measuring Pro-Renin Levels:

Prorenin can be measured indirectly by performing a renin assay after converting prorenin to renin (proteolytic or nonproteolytic). (Clin Chem. 1996; 42: 1051-1063). The results of this assay will reflect total renin levels, that is, the levels of prorenin plus renin. Subtracting the renin level from the total renin level is then a measure of prorenin.

Direct assays now exist for measuring pro-renin levels. For example, the Human Prorenin ELISA assay (available from BioVendor Research and Diagnostic Products) is a human prorenin direct assay intended for the quantitative determination of prorenin in biological fluids. Active renin will not be detected by this assay.

In this assay, prorenin is measured directly by ELISA without pretreatment of samples or conversion to renin. Human prorenin will bind to the capture antibody coated on a microtiter plate. Following appropriate washing steps, anti-human prorenin primary antibody binds to the captured protein. Only prorenin and not active renin will be detected by the primary antibody. Excess antibody is washed away and bound primary antibody is then reacted with the secondary antibody conjugated to horseradish peroxidase. TMB substrate is used for color development at 450 nm. A standard calibration curve is prepared along with the samples to be measured using dilutions of prorenin. The amount of color development is directly proportional to the concentration of prorenin in the sample.

Other direct and indirect assays used to measure prorenin levels are well know to one skilled in the art.

Measuring Aldosterone

Aldosterone assays can be performed on a blood sample or on a 24-hour urine specimen. Several factors, including diet, posture (upright or lying down), and time of day that the sample is obtained may alter aldosterone levels. Blood samples are affected by short-term fluctuations. A urine specimen collected over an entire 24-hour period improves the accuracy of the test.

Numerous kits are commercially available and widely used to perform aldosterone assays, including but not limited to the following: Human Aldosterone EIA Kit from Alpco Diagnostics, Human Aldosterone LIA Kit from Alpco Diagnostics, Aldosterone EIA Kit Assay from Designs/Stressgen Bioreagents, Aldosterone EIA Kit Assay from Designs/Stressgen Bioreagents, Human Aldosterone ELISA Kit from BioVendor Laboratory Medicine, Inc.

Measuring TGF-β

Transforming growth factor beta 1, also known as TGFbeta1, TGF beta, and TGF-β. The inactive form consists of a homodimer non-covalently linked to a latency-associated peptide homodimer. The active form is a homodimer of mature TGF-β that is disulfide linked. The precursor is cleaved into mature TGF-beta1 and the latency-associated peptide. Many cells can synthesize TGF-β, which is secreted to function in proliferation, differentiation, transformation, signaling and apoptosis. This cytokine has been implicated in many diseases including diabetes, renal disease, chronic pulmonary obstructive disease, as well as many cancers including prostate, and colon.

Numerous kits are commercially available and widely used to perform TGF-β assays. For example, the Human TGF-β1 ELISA kit from Anogen, the TGF-beta1 EIA Kit from Assay Designs/Stressgen, and the Human TGF-b1 ELISA Kit from Immuno-Biological Laboratories. Kits such as these are used for the in vitro quantitative determination of human transforming growth factor beta 1 (TGF-β1) concentrations in serum, plasma, cell culture supernatant, and other biological fluids.

Measuring Vitamin D Levels

Vitamin D is a fat-soluble vitamin naturally present in certain foods and available as a dietary supplement. The body can also produce Vitamin D endogenously from ultraviolet rays. Vitamin D obtained from sun exposure, food, and supplements, however, is biologically inactive and must undergo two hydroxylations before it converts into a physiologically active form. The first hydroxylation occurs in the liver, converting vitamin D to 25-hydroxyvitamin D [25(OH)D], also known as calcidiol. The second hydroxylation occurs primarily in the kidney and forms the physiologically active 1,25-dihydroxyvitamin D [1,25(OH)₂D] form, also known as calcitriol.

Vitamin D has many salubrious effects on human health, including modulation of neuromuscular and immune function and reduction of inflammation. Many genes encoding proteins that regulate cell proliferation, differentiation, and apoptosis are modulated in part by vitamin D. Vitamin D also enhances calcium absorption in the gut and helps to maintain adequate serum calcium and phosphate concentrations, which promotes healthy mineralization of bone and prevents hypocalcemic tetany.

Serum concentration of 25(OH)D is a key indicator of vitamin D levels. It reflects vitamin D produced cutaneously, as well as that obtained from food and supplements. Importantly, it also has a fairly long circulating half-life (15 days). However, serum 25(OH)D levels do not indicate the amount of vitamin D stored in other body tissues. Circulating 1,25(OH)₂D is generally not a good indicator of vitamin D status because it has a short half-life (15 hours) and serum concentrations are closely regulated by parathyroid hormone, calcium, and phosphate. Levels of 1,25(OH)₂D do not typically decrease until vitamin D deficiency has become severe.

Vitamin D deficiency has been studied at length, and there are now well accepted ranges correlated with Vitamin D deficiency. Specifically, serum concentrations of 25(OH)D below 10-11 ng/mL (25-27.5 nmol/L) are associated with vitamin D deficiency, and concentrations this low are generally considered inadequate for bone and overall health. Conversely, concentrations greater than or equal to 15 (ng/mL (37.5 nmol/L) are generally considered adequate for bone and overall health in healthy individuals.

Serum concentration of 25(OH)D consistently above 200 ng/mL (500 nmol/L), however, are considered potentially toxic, possibly leading to hypercalcemia and hyperphosphatemia, although human data is limited. In an animal model, concentrations ≦400 ng/mL (≦1,000 nmol/L) demonstrated no toxicity.

### Representative Examples

Existing and ongoing in vivo and in vitro studies have shown the efficacy of the pharmaceutical compositions and treatment regimes described herein. For example, combining a statin, fibrate, an aldosterone-antagonist, and optionally a thiazolidinedione, Vitamin D, and/or calcium to treat the adverse physiological conditions discussed above.

A. Combination Therapy

Subjects received one or more of a combination therapy of atorvastatin (Lipitor®), gemfibrozil (Lopid®), pioglitazone (ACTOS®), and spironolactone (Aldactone®), Vitamin D, and Calcium. Markers of inflammation and end-organ damage were assessed and monitored throughout the studies.

### Combination Therapy

| | | |
|---|---|---|
| Statin (Lipitor ®) | 600 | mg BID |
| Gemfibrozil (Lopid ®) | 20 | mg QD |
| Pioglitazone (ACTOS ®) and | 45 | mg QD |
| Spironolactone (Aldactone ®) | 25 | mg BID |
| (OR Eplerenone (INSPRA ®) if | 50 | mg QD |
| breast tenderness is troublesome) | | |
| Vitamin D | 1,000-30,000 | (D₃) |
| Calcium | 1000 | mg (twice |
| | | daily) |

Following administration of combination therapy to a subject, remarkable reductions in the level of aldosterone were observed. Normal aldosterone levels are approximately 30 ng/dL in healthy subjects. Diabetics typically have substantially elevated aldosterone levels. Because aldosterone production is stimulated via both a genomic and non-genomic pathway (See Nephrol Dial Transplant (2003) 18: 1693-1695), treatment with spironolactone (a mineralocorticoid receptor blocker) will nevertheless increase the levels of aldosterone. Often, administration of spironolactone to diabetic patients will increase aldosterone levels 400% or more. However, in one representative patient, a diabetic, aldosterone levels were substantially reduced (to a mere 9 ng/DL).

B. Unilateral Urethral Obstruction Experiment

In one study, three groups were examined following the obstruction of one ureter. The other ureter and kidney were left unobstructed. The control group received no treatment. A second group received an ACE inhibitor, while a third group received a statin and a gemfibrozil.

After treatment, the following results were observed. The control group had 100% relative kidney damage, compared to 50% damage in the second group (treated with an ACE inhibitor). The third group, however, treated with the combination of a statin and a gemfibrozil, had only 10% relative kidney damage. This was confirmed by measuring the levels of (pro)renin mRNA and AT₁ receptor, which were lowered substantially in the third group. The results suggest that the combination of a statin and a gemfibrozil suppresses both the circulatory and tissue RAS, a critical finding that supports the efficacy of the pharmaceutical treatment regime claimed herein.

C. Treatment of Diabetic Macular Edema

In another study, three patients with diabetic macular edema were treated with a statin and a gemfibrozil. Macular edema is characterized by the swelling of the retina in patients with diabetes mellitus, which results from the leaking of fluid from blood vessels within the macula. In diabetics, it is often accompanied by late onset neovascularization (of the eye), a condition which often results in blindness.

Two patients remained in the study, and up to a 95% reduction in neovascularization was observed; improved vision was also observed in these subjects. This was determined by monitoring the levels of vascular endothelial growth factor (VEGF) and the reduction of blood vessels in the vitreous. The third patient did not remain in the study, and subsequently went blind. Thus, although the sample population was of limited size, the study showed 100% efficacy.

1-55. (canceled)

56. A pharmaceutical composition comprising a fibrate anti-hyperlipidemic agent, a MG-CoA reductase inhibitor, an aldosterone antagonist and a pharmaceutically acceptable carrier.

57. The composition of claim 56, further comprising a thiazolidinedione (TZD) or a Angiotensin II Receptor Antagonists (AIIRA), wherein the fibrate anti-hyperlipidemic agent, the MG-CoA reductase inhibitor, and the aldosterone antagonist are in an amount sufficient to reduce inflammation and/or systemic endotoxemia in a human subject.

58. The composition of claim 56, further comprising Vitamin D and calcium, wherein the Vitamin D is selected from the group consisting of Vitamin D2 and Vitamin D3.

59. The composition of claim 56, wherein the fibrate anti-hyperlipidemic agent is between 100-1200 mg; the MG-CoA reductase inhibitor is between 5-80 mg; and the aldosterone antagonist is between 5-100 mg.

60. The composition of claim 59, wherein the fibrate anti-hyperlipidemic agent is gemfibrozil, the MG-CoA reductase inhibitor is a statin, and the aldosterone antagonist is a spironolactone or eplerenone.

61. The composition of claim 60, wherein the gemfibrozil is between 400-800 mg; the statin is between 10-30 mg; the spironolcatone is between 10-75 mg or eplereonone 15-70 mg.

62. The composition of claim 57, wherein the TZD is pioglitazone and the AIIRA is a telemisartan.

63. The composition of claim 62, wherein the pioglitazone is between 35-55 mg.

64. The composition of claim 58, wherein the Vitamin D3 is between 5,000 IU and 20,000 IU; Vitamin D2 is between 25,000 IU and 42,500 IU; and calcium is between 1,000 mg and 3,000 mg.

65. The composition of claim 56, wherein the fibrate anti-hyperlipidemic agent is 5-(2,5-dimethylphenoxy)-2,2-dimethylpentanoic acid; the MG-CoA reductase inhibitor is (3R,5R)-7-[2-(4-fluorophenyl)-3-phenyl-4-(phenylcarbamoyl)-5-(propan-2-yl)-1H-pyrrol-1-yl]-3,5-dihydroxyheptanoic acid; and the aldosterone antagonist is either 7α-Acetylthio-3-oxo-17α-pregn-4-ene-21,17-carbolactone or pregn-4-ene-7,21-dicarboxylic acid, 9,11-epoxy-17-hydroxy-3-oxo, γ-lactone, methyl ester (7α, 11α, 17α).

66. The composition of claim 65, further comprising 5-(4-[2-(5-ethylpyridin-2-yl)ethoxy]benzyl)thiazolidine-2,4-dione.

67. A pharmaceutical treatment regime for reducing inflammation and/or systemic endotoxemia in a subject, the regime comprising administering to a subject at least the following pharmacologically active ingredients within a 24 hour period: a fibrate anti-hyperlipidemic agent, a MG-CoA reductase inhibitor, an aldosterone antagonist, and optionally, a pharmaceutically acceptable carrier, wherein the pharmacologically active ingredients are in an amount sufficient to reduce inflammation and/or endotoxemia in the subject.

68. The pharmaceutical treatment regime of claim 67, wherein the pharmacologically active ingredients are in an amount to reduce the level of at least one pro-inflammatory marker selected from the group consisting of: prorenin, renin, renin mRNA, aldosterone, and TGF-β.

69. The pharmaceutical treatment regime of claim 68, wherein the treatment regime is useful in treating eye disease, kidney disease or end-organ injury after surgery.

70. The pharmaceutical treatment regime of claim 68, wherein the subject is a human.

71. The treatment regime of claim 67, further comprising administering a thiazolidinedione (TZD) or a Angiotensin II Receptor Antagonists (AIIRA).

72. The treatment regime of claim 67, further comprising administering Vitamin D and calcium, wherein the Vitamin D is selected from the group consisting of Vitamin D2 and Vitamin D3.

73. The treatment regime of claim 67, wherein the fibrate anti-hyperlipidemic agent is between 100-1200 mg; the MG-CoA reductase inhibitor is between 5-80 mg; and the aldosterone antagonist is between 5-100 mg.

74. The treatment regime of claim 73, wherein the fibrate anti-hyperlipidemic agent is gemfibrozil, the MG-CoA reductase inhibitor is a statin, and the aldosterone antagonist is a spironolactone or eplerenone.

75. The treatment regime of claim 74, wherein the gemfibrozil is between 400-800 mg; the statin is between 10-30 mg; the spironolcatone is between 10-75 mg or eplereonone 15-70 mg.

76. The treatment regime of claim 75, wherein the TZD is pioglitazone and the AIIRA is a telemisartan.

77. The treatment regime of claim 76, wherein the pioglitazone is between 35-55 mg; the Vitamin D3 is between 10,000 IU and 15,000 IU; the Vitamin D2 is between 25,000 IU and 42,500 IU; and the calcium is between 1,000 mg, and 3,000 mg.

78. The treatment regime of claim 67, wherein the fibrate anti-hyperlipidemic agent is 5-(2,5-dimethylphenoxy)-2,2-dimethylpentanoic acid; the MG-CoA reductase inhibitor is (3R,5R)-7-[2-(4-fluorophenyl)-3-phenyl-4-(phenylcarbamoyl)-5-(propan-2-yl)-1H-pyrrol-1-yl]-3,5-dihydroxyheptanoic acid; and the aldosterone antagonist is either 7α-Acetylthio-3-oxo-17α-pregn-4-ene-21,17-carbolactone or pregn-4-ene-7,21-dicarboxylic acid, 9,11-epoxy-17-hydroxy-3-oxo, γ-lactone, methyl ester (7α, 11α, 17α).

79. The treatment regime of claim 78, further comprising 5-(4-[2-(5-ethylpyridin-2-yl)ethoxy]benzyl)thiazolidine-2,4-dione.

80. A pharmaceutical treatment regime for lowering the C-reactive protein (CRP) level in a human patient's blood, the regime comprising administering to a subject at least the following pharmacologically active ingredients within a 24 hour period: a fibrate anti-hyperlipidemic agent, a MG-CoA reductase inhibitor, an aldosterone antagonist, and optionally a pharmaceutically acceptable carrier, wherein the pharmacologically active ingredients are in an amount sufficient to lower the C-reactive protein (CRP) level in the patient's blood.

81. A kit comprising an effective amount of the following pharmacologically active ingredients: a fibrate anti-hyperlipidemic agent, a MG-CoA reductase inhibitor, an aldosterone antagonist and optionally a pharmaceutically acceptable carrier and instructions describing the pharmaceutical treatment regime of claim 12.

82. The kit of claim 81, further comprising a thiazolidinedione (TZD) or a Angiotensin II Receptor Antagonists (AIIRA).

83. The kit of claim 81, further comprising Vitamin D and calcium, wherein the Vitamin D is selected from the group consisting of Vitamin D2 and Vitamin D3.

84. The kit of claim 81, wherein the fibrate anti-hyperlipidemic agent is 5-(2,5-dimethylphenoxy)-2,2-dimethylpentanoic acid; the MG-CoA reductase inhibitor is (3R,5R)-7-[2-(4-fluorophenyl)-3-phenyl-4-(phenylcarbamoyl)-5-(propan-2-yl)-1H-pyrrol-1-yl]-3,5-dihydroxyheptanoic acid; and the aldosterone antagonist is either 7α-Acetylthio-3-oxo-17α-pregn-4-ene-21,17-carbolactone or pregn-4-ene-7,21-dicarboxylic acid, 9,11-epoxy-17-hydroxy-3-oxo, γ-lactone, methyl ester (7α, 11α, 17α).
